(19) United States
(12) Patent Application Publication
(10) Pub. No.: US 2011/0152664 A1
(43) Pub. Date: Jun. 23, 2011

(54) MEASURING DEVICE AND A METHOD FOR DETERMINING MOVEMENT IN A TISSUE

(75) Inventor: Martin Leibfritz, Muenchen (DE)

(73) Assignee: ROHDE & SCHWARZ GMBH & CO. KG, Muenchen (DE)

(21) Appl. No.: 12/995,981

(22) PCT Filed: Jun. 2, 2009
(86) PCT No.: PCT/EP2009/003923

§ 371 Date: Feb. 3, 2011

(30) Foreign Application Priority Data
Jun. 2, 2008 (DE) 10 2008 026 434.2
Dec. 16, 2008 (DE) 10 2008 062 485.3

(51) Int. Cl. A61B 5/0265

(52) U.S. Cl. 600/407

(57) ABSTRACT

A measuring device contains a microwave transmitter, a microwave receiver and a control device. The control device controls the microwave transmitter in such a manner that the latter transmits a microwave signal into a tissue. The tissue contains moving constituents. The tissue controls the microwave signal. The moving constituents of the tissue change the frequency of the microwave signal. The control device controls the microwave receiver in such a manner that the latter receives the scattered and/or frequency-changed microwave signal. The control device determines from the received microwave signal a movement of the moving constituents of the tissue.

The invention of relates to a measuring device and a measuring method for determining movement within a tissue, in particular, a microwave tomography device and a method for microwave tomography with Doppler analysis.

Conventionally, ultrasound investigations are implemented for the detection of movements in tissue. For example, DE 196 19 808 A1 discloses an ultrasound diagnosis system which registers and displays movements. However, these allow only a poor image quality. Furthermore, they demand a high level of experience from the operating personnel. Moreover, in view of the poor image quality, small blood vessels cannot be reliably identified. However, for the diagnosis of tumorous diseases, such capillaries are essential, because many tumours are surrounded by such capillaries.

The invention is based upon the object of providing a method and a measuring device, which determine movements within tissue with a good image quality and place low demands on the operating personnel.

The object is achieved according to the invention for the measuring device by the features of the independent claim 1 and for the method by the features of the independent claim 9. Advantageous further developments form the subject matter of the dependent claims relating back to these claims.

A measuring device according to the invention contains a microwave transmitter, a microwave receiver and a control device. The control device controls the microwave transmitter in such a manner that the latter transmits a microwave signal into a tissue. The tissue contains moving constituents. The tissue scatters the microwave signal. The moving constituents of the tissue change the frequency of the microwave signal. The control device controls the microwave receiver in such a manner that the latter receives the scattered and/or frequency-changed microwave signal. The control device determines from the received microwave signal a movement of the moving constituents of the tissue. Accordingly, movements within the tissue are detected. The results of the investigation are independent of the qualifications of the operating personnel.

The measuring device preferably contains at least one antenna. By means of the at least one antenna, the microwave signal transmitted by the microwave transmitter is preferably directed successively to given locations within the tissue and/or the microwave signal received by the microwave receiver is preferably directed to given locations within the tissue. The control device preferably determines from the received microwave signals of the given locations a microwave tomography of the tissue. A high local resolution can be achieved in this manner.

The measuring device advantageously contains a display device. The control device advantageously controls the display device. By preference, the control device displays a direction and strength of the movement on the display device. Accordingly, the results can be readily further processed. In particular, a determination of tumours in tissue is facilitated in this manner.

The control device preferably displays the microwave tomography on the display device. In this manner, additional information about the tissue can be readily exploited.

The control device advantageously displays the microwave tomography and the movement in a superimposed manner on the display device. By preference, the control device displays the movement in a colour-coded manner on the display device. Accordingly, the microwave tomography and the movements within the tissue can be readily compared. In particular, a reference of the precise location of the movements is possible in this manner.

The moving constituents of the tissue are preferably blood. The control device preferably detects blood vessels and/or tumours. In this manner, a determination of tissue changes can also be implemented by personnel with low qualifications.

The transmitted microwave signal is advantageously a frequency sweep. The microwave receiver advantageously receives on a fixed frequency. Accordingly, a very simple receiver can be used.

As an alternative, the transmitted microwave signal is largely a mono-frequency. The microwave receiver preferably receives by means of a frequency sweep. In this manner, a precise result can be achieved.

The invention is described by way of example below on the basis of the drawings, which present an advantageous exemplary embodiment of the invention. The drawings are as follows:

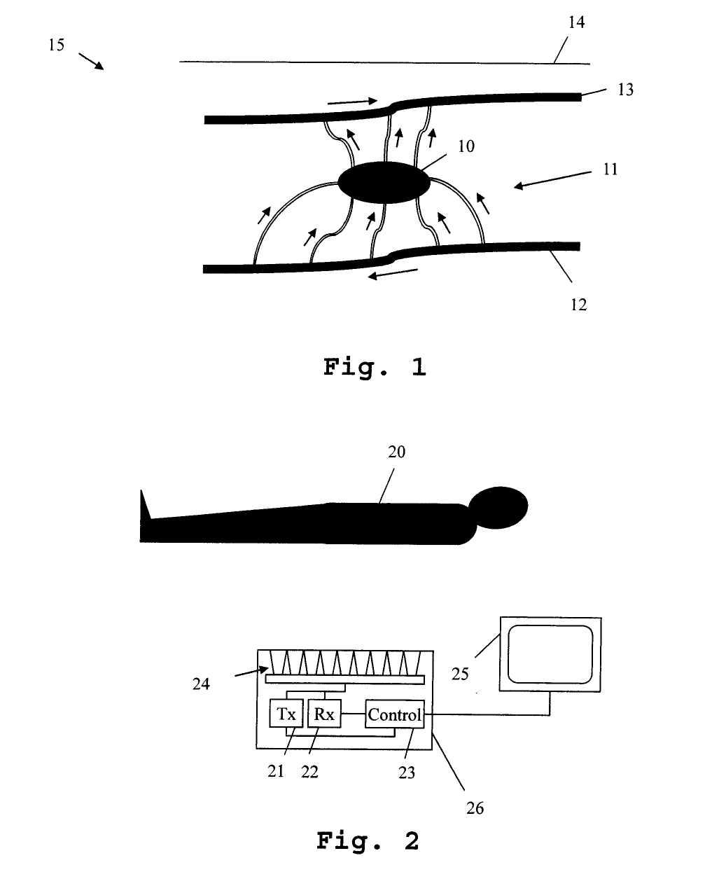

FIG. 1 shows a sectional view of an exemplary tumour tissue;

FIG. 2 shows a first exemplary embodiment of the measuring device according to the invention;

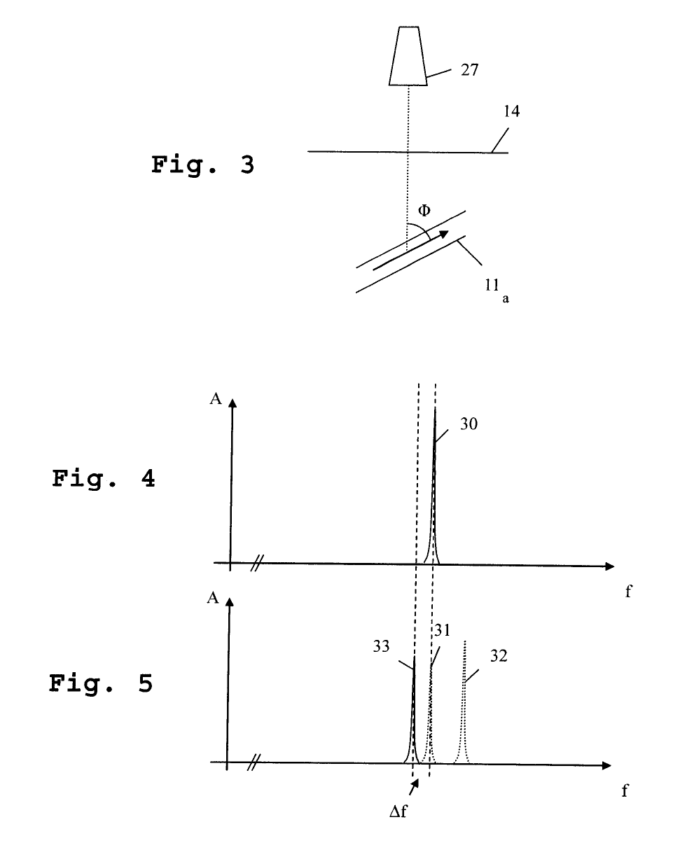

FIG. 3 shows a second exemplary embodiment of the measuring device according to the invention;

FIG. 4 shows a microwave signal transmitted by a third exemplary embodiment of the device according to the invention in a frequency diagram;

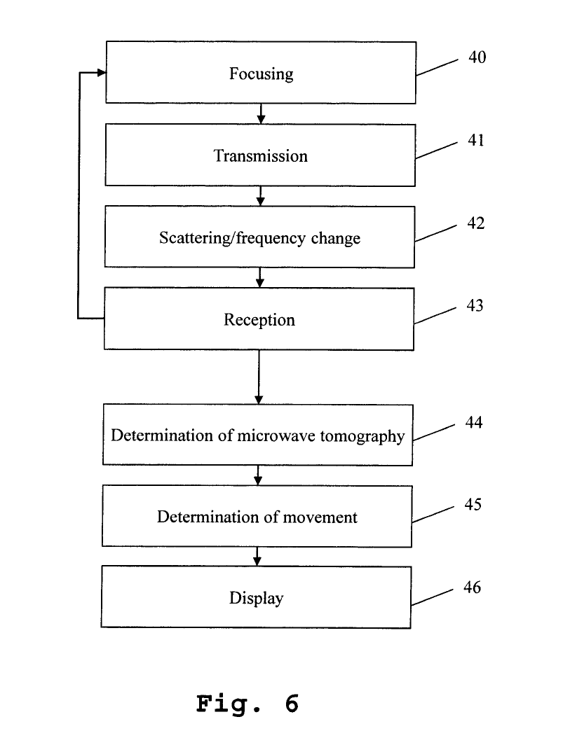

FIG. 5 shows exemplary Doppler shifts in a frequency diagram; and

FIG. 6 shows a flow diagram of an exemplary embodiment of the method according to the invention.

Initially, the general problem and the basic method of functioning of the device according to the invention and the method according to the invention are explained on the basis of FIG. 1. By means of FIGS. 2-5, the structure and function of the device according to the invention are then described on the basis of the various embodiments. Finally, with reference to FIG. 6, the functioning of the method according to the invention is shown. The presentation and description of identical elements in similar diagrams has not been repeated in some cases.

FIG. 1 shows a sectional view of an exemplary tumour tissue. A tissue 15 is limited by a tissue surface 14. The tissue 15 contains an artery 12, a vein 13, a tumour 10 and a plurality of capillaries 11. The tumour 10 is connected by means of a plurality of capillaries 11 both to the artery 12 and also to the vein 13.

In this context, blood flows from the artery 12 through the capillaries 11 to the tumour 10, and from there through further capillaries 11 to the vein 13. At each location within a capillary 11, the artery 12 or the vein 13, the blood provides a flow direction. The remainder of the tissue remains largely at rest.

Tumours send out messenger substances, which trigger vascular proliferation. This leads to an increased growth of capillaries in their proximity. On the basis of this increased presence of blood-carrying capillaries in the proximity of the tumours, these can be detected, by determining regions of concentrated movement of blood.

FIG. 2 shows a first exemplary embodiment of the measuring device according to the invention. A microwave transmitter 21, a microwave receiver 22, a control device 23 and an antenna array 24 are arranged in a housing 26. The control device 23 is connected, in this case, to a display device 25 arranged outside the housing 26. An arrangement of the display device 25 inside the housing 26 is also possible. Furthermore, the control device 23 is connected to the microwave transmitter 21 and the microwave receiver 22. The microwave transmitter 21 and the microwave receiver 22 are each connected to the antenna array 24. Instead of an antenna array 24, several individual antennas or also only a single antenna can also be used. In order to achieve a local resolution with only a single antenna, several antenna positions relative to the tissue are required. This is achieved through a movement of the antenna relative to the tissue.

For the implementation of a measurement, the control device 23 controls the microwave transmitter 21 in such a manner that the latter transmits a microwave signal by means of the antenna array 24 into a tissue, here, of a patient 20. In this context, the microwave signal is a frequency sweep. That is to say, a plurality of different, largely mono-frequency microwave signals is transmitted over a defined period. The plurality of mono-frequency microwave signals accordingly covers a given frequency range. The microwave signals penetrate into the tissue and are scattered by the latter. Moving elements within the tissue, for example, flowing blood, cause a frequency change of the microwave signal as a result of the Doppler effect.

By means of the antenna array 24, the microwave receiver 22 receives the microwave signals scattered and frequency-changed by the tissue. In this case, the microwave receiver 22 receives only on a single frequency. As an alternative to the transmission of a frequency sweep and the reception of a fixed frequency, the transmission of a fixed frequency and reception of a frequency sweep is also possible. A particularly good accuracy can be achieved, if a frequency sweep is transmitted and a frequency sweep is received for each individual frequency of the transmitted frequency sweep.

In each case, the control device 23 determines movements within the tissue from the microwave signals transmitted and received at a given timing point. Furthermore, the control device 23 determines a microwave tomography of the tissue. A local resolution of the movements within the tissue and the microwave tomography is achieved through a sequential exploitation of the antenna array 24. That is to say, by means of a plurality of transmission positions and reception positions, a plurality of localisation points in the tissue is registered.

To achieve a further increase in accuracy, a two-stage measurement method can be used. Accordingly, for the entire tissue, it is first determined in which regions movements occur and approximately how large the movements are. In a second step, these regions are investigated in a targeted manner for movements within the given velocity range. A very high velocity resolution is achieved in this manner.

The microwave tomography and the movements within the tissue are displayed on the display device 25 in a superimposed manner.

Additionally, a detection of tissue alterations can be implemented. For example, tumours can be detected in an automated manner on the basis of the numerous blood vessels surrounding them.

FIG. 3 shows a second exemplary embodiment of the measuring device according to the invention. Here, only a single antenna 27, the tissue surface 14 and a single capillary 11a are shown. The antenna 27, in this context, transmits a microwave signal in the direction towards the tissue. Here, the flow direction of the blood within the capillary 11a forms an angle Φ relative to the Poynting vector of the incident wave. On the basis of the Doppler effect, the frequency change is obtained as follows from the flow velocity v of the blood and the angle Φ:

Δf=2*f*v/c₀* cos Φ.

The frequency change is therefore proportionally larger, the higher the flow velocity of the blood is, and the more parallel relative to the incident wave the flow direction of the blood is. Movements within the tissue which extend exactly perpendicular to the incident wave cause no frequency change and cannot therefore be detected. For practical purposes, however, this case is not relevant because of the different antenna positions.

FIG. 4 illustrates a microwave signal 30 transmitted from a third exemplary embodiment of the device according to the invention. A detail from a microwave signal transmitted from the microwave transmitter plotted against the frequency is illustrated. It provides substantially only a single frequency component.

In the case of an excitation of a given location within the tissue with this single frequency component, the signal is scattered by the tissue and changed in its frequency by a movement of the location within the tissue relative to the antenna.

FIG. 5 shows exemplary Doppler shifts in a frequency diagram. If a location within the tissue which is disposed at rest is irradiated with the microwave signal 30 from FIG. 4, an attenuated signal 31 is received. Because the location within the tissue is disposed at rest, no change of the frequency of the microwave signal 30 occurs. However, if the location within the tissue is not disposed at rest, a frequency change occurs. If the location within the tissue is, for example, a portion of a blood-carrying capillary, of which the direction of flow is orientated against the direction of the antenna, a microwave signal 33 with reduced frequency is received. By contrast, if the direction of flow is towards the antenna, a microwave signal 32 of increased frequency is received. The angle Φ or respectively the flow velocity v can be inferred from the changing frequency Δf.

FIG. 6 shows an exemplary embodiment of the method according to the invention. In a first step 40, a focusing on a given location within the tissue is implemented. This can occur through a movement of an individual transmission antenna. A focusing by means of an antenna array is also possible. Alongside a focusing of this kind in the transmitter, a focusing with a homogeneous excitation is also possible in the receiver. Accordingly, a single, moving reception antenna and also an antenna array can be used.

In a second step 41, a microwave signal is transmitted into the given location of the tissue. In a third step 42, a microwave signal undergoes a scattering by the tissue. If the location within the tissue describes a movement relative to the antenna position, the microwave signal additionally undergoes a frequency change. In a fourth step 43, the scattered and/or frequency-changed microwave signal is received.

The steps 40-43 are repeated in rapid succession for a plurality of locations within the tissue. In a fifth step 44, when all of the locations to be investigated within the tissue have been worked through, a microwave tomography of the tissue is determined from the scatterings of the microwave signals. In a sixth step 45, movements within the tissue are determined from the frequency changes of the microwave signals.

Finally, in a seventh step 46, the microwave tomography and also the movements within the tissue are displayed in a superimposed manner. Displaying the microwave tomography in grey scales with a display of the movements within the tissue in colour is another possibility in this context. In this context, different colour gradations encode the velocity or the direction of the movement.

Optionally, an additional, automatic detection of tissue changes can be implemented. Accordingly, tissue with a particularly strong circulation of blood, which often occurs in the environment of tumours, can be inferred from high movement concentrations. In particular, high movement concentrations of different directions indicate a fine mesh of capillaries. Such detected tissue changes are displayed in addition to the microwave tomography and the movements within the tissue.

The invention is not restricted to the illustrated exemplary embodiment. As already mentioned, different tissue changes can be detected. Movements of different tissue constituents can also be determined. Alongside the movement of blood, the movement of other body fluids and also the movement of solid tissue constituents, for example, of the heart muscle, can be determined. All of the features described or shown in the drawings can be advantageously combined with one another as required within the framework of the invention.

1. A measuring device with a microwave transmitter, a microwave receiver and a control device,
wherein the control device controls the microwave transmitter in such a manner that the latter transmits a microwave signal into a tissue,
wherein the tissue contains moving constituents,
wherein the tissue scatters the microwave signal,
wherein the moving constituents of the tissue change the frequency of the microwave signal,
wherein the control device controls the microwave receiver in such a manner that the latter receives the scattered and/or frequency-changed microwave signal,
wherein the control device is formed in such a manner that it determines from the received microwave signal a movement of the moving constituents of the tissue and detects tissues changes in an automated manner.

2. The measuring device according to claim 1, characterized in that
the measuring device contains at least one antenna,
that, by means of the at least one antenna, the microwave signal transmitted by the microwave transmitter is directed successively to given locations within the tissue, and/or the microwave signal received by the microwave receiver is directed to given locations within the tissue,
and that the control device determines from the received microwave signals of the given locations a microwave tomography of the tissue.

3. The measuring device according to claim 1, characterized in that
the measuring device contains a display device,
that the control device controls the display device, and
that the control device displays a direction and strength of the movement on the display device.

4. The measuring device according to claim 2, characterized in that
the control device displays the microwave tomography on the display device.

5. The measuring device according to claim 4, characterized in that
the control device displays the microwave tomography and the movement in a superimposed manner on the display device, and
that the control device displays the movement in a color-coded manner on the display device.

6. The measuring device according to claim 1, characterized in that
the moving constituents of the tissue are blood, that the control device detects blood vessels and/or tumors.

7. The measuring device according to claim 1, characterized in that
the transmitted microwave signal is a frequency sweep through different frequencies, and
that the microwave receiver receives at a fixed frequency.

8. The measuring device according to claim 1, characterized in that
the transmitted microwave signal is largely a mono-frequency, and
that the microwave receiver receives at different frequencies by means of a frequency sweep.

9. A method for determining movement within a tissue with a microwave transmitter, a microwave receiver and a control device,
wherein the control device controls the microwave transmitter and the microwave receiver,
wherein the tissue contains moving constituents,
the method comprising:
transmitting a microwave signal into the tissue by the microwave transmitter;
scattering of the microwave signal by the tissue;
changing of the frequency of the microwave signal by the moving constituents of the tissue;
receiving the scattered and/or frequency-changed microwave signal by the microwave receiver; and
determining a movement of the moving constituents of the tissue from the received microwave signal by the control device and automatic detection of tissue changes.

10. The method according to claim 9, characterized in that,
by means of at least one antenna, the microwave signal transmitted by the microwave transmitter is directed successively to given locations within the tissue and/or the microwave signal received by the microwave receiver is directed to given locations within the tissue, and
that a microwave tomography is determined by the control device from the received microwave signals of the given locations of the tissue.

11. The method according to claim 9, characterized in that
a display device is controlled by the control device, and
that a direction and strength of the movement is displayed by the control device on the display device.

12. The method according to claim 10, characterized in that
the microwave tomography is displayed on the display device.

13. The method according to claim 12, characterized in that
the microwave tomography and the movement are displayed in a superimposed manner on the display device, and
that the movement is displayed in a color-coded manner on the display device.

14. The method according to claim 9, characterized in that
the moving constituents of the tissue are blood,
that blood vessels and/or tumors are detected by the control device.

15. The method according to claim 9, characterized in that
a frequency sweep through different frequencies is transmitted by the microwave transmitter as the microwave signal, and
that microwave signals of a fixed frequency are received by the microwave receiver.

16. The method according to claim 9, characterized in that
a mono-frequency microwave signal is transmitted by the microwave transmitter, and
that the microwave receiver receives on different frequencies by means of a frequency sweep.
